SEQUENCE LISTING (29 sequences)

SEQ ID NO: 1 - DNA (other nucleic acid), 30 bp; single-stranded, linear
  description: /desc = “Primer”
cattatgctgagtgatatctttttttttvv

SEQ ID NO: 2 - DNA (other nucleic acid), 30 bp; single-stranded, linear
  description: /desc = “Primer”
cattatgctgagtgatatctttttttttaa

SEQ ID NO: 3 - DNA (other nucleic acid), 30 bp; single-stranded, linear
  description: /desc = “primer”
cattatgctgagtgatatctttttttttac

SEQ ID NO: 4 - DNA (other nucleic acid), 30 bp; single-stranded, linear
  description: /desc = “primer”
cattatgctgagtgatatctttttttttag

SEQ ID NO: 5 - DNA (other nucleic acid), 30 bp; single-stranded, linear
  description: /desc = “primer”
cattatgctgagtgatatctttttttttca

SEQ ID NO: 6 - DNA (other nucleic acid), 30 bp; single-stranded, linear
  description: /desc = “primer”
cattatgctgagtgatatctttttttttcc

SEQ ID NO: 7 - DNA (other nucleic acid), 30 bp; single-stranded, linear
  description: /desc = “primer”
cattatgctgagtgatatctttttttttcg

SEQ ID NO: 8 - DNA (other nucleic acid), 30 bp; single-stranded, linear
  description: /desc = “primer”
cattatgctgagtgatatctttttttttga

SEQ ID NO: 9 - DNA (other nucleic acid), 30 bp; single-stranded, linear
  description: /desc = “primer”
cattatgctgagtgatatctttttttttgc

SEQ ID NO: 10 - DNA (other nucleic acid), 30 bp; single-stranded, linear
  description: /desc = “primer”
cattatgctgagtgatatctttttttttgg

SEQ ID NO: 11 - DNA (other nucleic acid), 26 bp; single-stranded, linear
  description: /desc = “primer”
attaaccctcactaaannnnnnnnnn

SEQ ID NO: 12 - DNA (other nucleic acid), 25 bp; single-stranded, linear
  description: /desc = “primer”
attaaccctcactaaatgctgggga

SEQ ID NO: 13 - DNA (other nucleic acid), 25 bp; single-stranded, linear
  description: /desc = “primer”
attaaccctcactaaatgctggagg

SEQ ID NO: 14 - DNA (other nucleic acid), 25 bp; single-stranded, linear
  description: /desc = “primer”
attaaccctcactaaatgctggtag

SEQ ID NO: 15 - DNA (other nucleic acid), 25 bp; single-stranded, linear
  description: /desc = “primer”
attaaccctcactaaatgctggtag

SEQ ID NO: 16 - DNA (other nucleic acid), 26 bp; single-stranded, linear
  description: /desc = “primer”
attaaccctcactaaagatctgactg

SEQ ID NO: 17 - DNA (other nucleic acid), 25 bp; single-stranded, linear
  description: /desc = “primer”
attaaccctcactaaatgctgggtg

SEQ ID NO: 18 - DNA (other nucleic acid), 25 bp; single-stranded, linear
  description: /desc = “primer”
attaaccctcactaaatgctgtatg

SEQ ID NO: 19 - DNA (other nucleic acid), 25 bp; single-stranded, linear
  description: /desc = “primer”
attaaccctcactaaatggagctgg

SEQ ID NO: 20 - DNA (other nucleic acid), 25 bp; single-stranded, linear
  description: /desc = “primer”
attaaccctcactaaatgtggcagg

SEQ ID NO: 21 - DNA (other nucleic acid), 26 bp; single-stranded, linear
  description: /desc = “primer”
attaaccctcactaaatgcaccgtcc

SEQ ID NO: 22 - DNA (other nucleic acid), 12 bp; single-stranded, linear
  description: /desc = “primer”
gatctgcggtga

SEQ ID NO: 23 - DNA (other nucleic acid), 24 bp; single-stranded, linear
  description: /desc = “primer”
agcactctccagcctctcaccgca

SEQ ID NO: 24 - DNA (other nucleic acid), 12 bp; single-stranded, linear
  description: /desc = “primer”
gatctgttcatg

SEQ ID NO: 25 - DNA (other nucleic acid), 24 bp; single-stranded, linear
  description: /desc = “primer”
accgacgtcgactatccatgaaca

SEQ ID NO: 26 - DNA (other nucleic acid), 12 bp; single-stranded, linear
  description: /desc = “primer”
gatcttccctcg

SEQ ID NO: 27 - DNA (other nucleic acid), 24 bp; single-stranded, linear
  description: /desc = “primer”
aggcaactgtgctatccgagggaa

SEQ ID NO: 28 - DNA (other nucleic acid), 27 bp; single-stranded, linear
  description: /desc = “primer”
gcaactaccatttggccactcaggaag

SEQ ID NO: 29 - DNA (other nucleic acid), 25 bp; single-stranded, linear
  description: /desc = “primer”
gcaaccaacccttctccaccagcag

(19) United States
(12) Patent Application Publication
(10) Pub. No.: US 2002/0037511 A1
(43) Pub. Date: Mar. 28, 2002

(54) Method for identifying genes

(76) Inventors: Paz Einat, Nes-Ziona (IL); Rami Skaliter, Nes-Ziona (IL); Orna Mor, Kiryat Ono (IL); Sylvie Luria, Nes-Ziona (IL); Nicholas Harris, Rehovot (IL); Zehava Grosman, Rehovot (IL)

Correspondence Address: Kohn & Associates, Suite 410, 30500 Northwestern Hwy., Farmington Hills, MI, 48334, US

(21) Appl. No.: 09/792,471

(22) Filed: Feb. 23, 2001

Related U.S. Application Data
(63) Continuation of application No. 09/309,862, filed on May 11, 1999, now abandoned.
(60) Provisional application No. 60/084,944, filed on May 11, 1998.

(51) Int. Cl. C12Q 1/68; C07H 21/02; C07H 21/04

(52) U.S. Cl. 435/6; 536/23.1

(57) ABSTRACT

A method for identifying genes regulated at the RNA level by cue-induced gene expression. The invention relates to the rapid isolation of differentially expressed or developmentally regulated gene sequences through analysis of mRNAs obtained from specific cellular compartments and comparing the changes in the relative abundance of the mRNA in these compartments as a result of applying a cue to the tested biological sample. The cellular compartments include polysomal and nonpolysomal fractions, nuclear fractions, cytoplasmic fractions, and spliceosomal fractions. Genes that are differentially expressed due to regulation on any one or more of a number of levels, may be characterized. Regulation levels include translational regulation, transcriptional regulation, mRNA stability regulation, and mRNA transport regulation. A method for identifying gene sequences coding for internal ribosome entry sites is also provided, which includes inhibiting 5′ cap-dependant mRNA translation in a cell, collecting a pool of mRNA from the cells, and differentially analyzing the pool of mRNA to identify genes with sequences coding for internal ribosome entry sites.

### CROSS REFERENCE TO RELATED APPLICATIONS

This application is a conversion of U.S. Provisional Patent Application Serial No. 60/084,944, filed May 11, 1998, and claims priority thereon.

### BACKGROUND OF THE INVENTION

1. Technical Field

The present invention relates to a method for identifying genes that are regulated at the RNA level. More specifically, the present invention relates to the rapid isolation of differentially expressed or developmentally regulated gene sequences through analysis of mRNAs obtained from specific cellular compartments. By comparing changes in the relative abundance of the mRNAs found in these compartments occurring as a result of application of a cue or stimulus to the tested biological sample, genes that are differentially expressed can be characterized.

2. Background Art

The identification and/or isolation of genes whose expression differs between two cell or tissue types, or between cells or tissues exposed to stress conditions, chemical compounds or pathogens, is critical to the understanding of mechanisms which underlie various physiological conditions, disorders, or diseases. Regulation of gene expression has been shown to play an important part in many biological processes including embryogenesis, aging, tissue repair, and neoplastic transformation. Regulation of gene expression can occur on a number of levels, including scriptional regulation, translational regulation, regulation of mRNA stability, regulation of mRNA transport, regulation by natural antisense mRNA and regulation by alternative splicing. However, while cases of genes thus regulated are reported in the literature, the gene discovery approaches followed to date have only examined changes in the ‘steady state’ levels of cellular mRNA by analysis of total cellular RNA.

A number of methods have been developed for the detection and isolation of genes which are activated or repressed in response to developmental, physiological, pharmacological, or other cued events. One particular method is described in U.S. Pat. No. 5,525,471 to Zeng, is subtractive hybridization. Subtractive hybridization is a particularly useful method for selectively cloning sequences present in one DNA or RNA population while absent in another, but is less sensitive to more subtle differences. The selective cloning is accomplished by generating single stranded complementary DNA libraries from both control cells/tissue (driver cDNA) and cell/tissue during or after a specific change or response being studied (tester cDNA). The two cDNA libraries are denatured and hybridized to each other resulting in duplex formation between the driver and tester EDNA strands. In this method, common sequences are removed and the remaining non-hybridized single-stranded DNA is enriched for sequences present in the experimental cell/tissue which is related to the particular change or event being studied. Davis et al., 1987).

Currently used methodologies to identify mRNAs encoding proteins which are being induced/reduced following a cue or stimulus rely on changes in steady state mRNA levels via screening of differentially expressed mRNAs. One such method for the identification of differentially expressed mRNAs is disclosed in U.S. Pat. No. 5,459,037 to Sutcliffe et al. According to this method, an mRNA population is isolated, double-stranded cDNAs are prepared from the mRNA population using a mixture of twelve anchor primers, the cDNAs are cleaved with two restriction endonucleases, and then inserted into a vector in such an orientation that they are anti-sense with respect to a T3 promotor within the vector. E. coli are transformed with the cDNA containing vectors, linearized fragments are generated from the cloned inserts by digestion with at least one restriction endonuclease that is different from the first and second restriction endonucleouseases and a cDNA preparation of the anti-sense cDNA transcripts is generated by incubating the linearized fragments with a T3 RNA polymerase. The cDNA population is divided into subpools and the first strand cDNA from each subpool is transcribed using a thermostable reverse transcriptase and one of sixteen primers. The transcription product of each of the sixteen reaction pools is used as a template for a polymerase chain reaction (PCR) with a 3′-primer and a 5′-primer and the polymerase chain reaction amplified fragments are resolved by electrophoresis to display bands representing the 3′-ends of the mRNAs present in the sample. This method is useful for the identification of differentially expressed mRNAs and the measurement of their relative concentrations. This type of methodology, however, is unable to identify mRNAs whose levels remain constant but whose translatability is variable or changes, or differences resulting from changes in mRNA transport from the nucleus to the cytoplasm.

Schena et al. developed a high capacity system to monitor the expression of many genes in parallel utilizing microarrays. The microarrays are prepared by high speed robotic printing of cDNAs on glass providing quantitative expression measurements of the corresponding genes (Schena et al., 1995). Differential expression measurements of genes are made by means of simultaneous, two color fluorescence hybridization. However, this method alone is of limited sensitivity and is insufficient for the identification of several types of regulation levels, including translationally regulated genes and mRNA transport regulation. The authors did not examine the use of special mRNA pools that enable direct assessment of transcriptional activity.

The use of a known inhibitor of hypusine formation, mimosime, was used to reversibly suppress the hypusine-forming deoxyhypusyl hydroxylase in cells while differentially displaying their polysomal versus non-polysomal mRNA populations (Hanauske-Abel et al., 1995) Utilizing this method, several species of mRNA were discovered which disappear and reappear, respectively, at polysomes in connection with inhibition and disinhibition of hypusine formation and which are thought to code for translationally controlled enzymes. This method only teaches the use of a known stimulating element (i.e., inducer or repressor) to identify translationally regulated genes. (This method does not provide a mechanism for the detection and/or identification of translationally regulated genes where the stimulating element is unknown). The use of differential display for gene discovery is fiery limited in terms of throughput and sensitivity and is prone to many artifices. The subject matter of this paper does not imply the use of polysomal mRNA pools as sources for probes for DNA chip analysis. This in fact requires special methodological improvements in order to obtain large amounts of high quality polysomal mRNA.

Generally, the translation of eukaryotic mRNAs is dependent upon 5′cap-mediated ribosome binding. Prior to translation, the ribosome small sub-unit (40S) binds to the 5′-cap structure on a transcript and then proceeds to scan along the mRNA molecule to the translation initiation site where the large sub-unit (60S) forms the complete ribosome initiation site. In most instances, the translation initiation site is the first AUG codon. This “scanning model” of translation initiation accommodates most eukaryotic mRNAs. A few notable exceptions to the “scanning model” are provided by the Picornavirus family. These viruses produce non-capped transcripts with long (600-1200 nucleotides) 5′-untranslated regions (UTR) which contain multiple non-translation initiating AUG codons. Because of the absence of a cap structure, the translational efficiency of these RNAs is dependent upon the presence of specific sequences within the untranslated regions (UTR) known as internal ribosome entry sites (IRES).

More recently, IRES containing mRNA transcripts have been discovered in non-viral systems such as the mRNA encoding for immunoglobulin heavy chain binding protein, the antenapedia gene in Drosophila, and the mouse Fgl-2 gene. These discoveries have promoted speculation for the role of cap-independent translation in the developmental regulation of gene expression during both normal and abnormal processes.

The discovery of the above-mentioned non-viral IRES containing mRNAs implies that eukaryotic IRES sequences could be more wide spread than has been previously realized. The difficulty in identifying eukaryotic IRES sequences resides in the fact that they typically cannot be identified by sequence homology. [Oh et al., 1993; Mountford et al., 1995; Macejak et al., 1991; Pelletier et ale, 1988; Vagner et al. 1995] It would, therefore, be advantageous to have a method for identifying IRES containing ERNA in order to identify translationally controlled genes operating via 5′-cap independent translation in order to ascertain and assess their association with both normal and abnormal processes.

Prior art methods have only concentrated on very narrow aspects of gene expression regulation and used methods which have many inherent limitations. Therefore, it would be desirable to have methods that allow us to expand the array of gene expression regulation levels and thus enable the isolation of biologically important genes.

### SUMMARY OF THE INVENTION

According to the present invention, methods are provided for identifying genes that may be regulated on a number of possible regulatory levels. Such methods include the steps of exposing cells or tissue to a cue or stimulus such as mechanical, chemical, toxic, pharmaceutical or other stress, hormones, physiological disorders or disease; fractionating the cells into compartments such as polysomes, nuclei, cytoplasm and spliceosomes; extracting the mRNA from these fractions, and subjecting the mRNA to differential analysis using accepted methodologies, such as gene expression array (GEM).

An example is provided which shows the use of RNA isolation from nuclei for isolating genes whose steady state levels show only minor changes, but which show high differential expression when detected by nuclear RNA probe. Most such genes are regulated at the transcriptional level. Another example is provided, of one type of regulation showing the use of polysomes isolated from cells/tissues to identify genes whose mRNA steady state levels do not change, but are highly increased in the polysomes after application of a stress cue. Such genes are regulated strictly on the translation level.

A subgroup of genes regulated on the translational level involves the existence of internal ribosome entry sites. A method is provided for identification of such genes, which includes inhibiting 5′cap-dependant mRNA translation in a cell, collecting a pool of mRNA from the cells, and differentially analyzing the pool of mRNA to identify genes with sequences coding for internal ribosome entry sites.

### BRIEF DESCRIPTION OF THE DRAWINGS

Other advantages of the present invention will be readily appreciated as &Le same becomes better understood by reference to the following detailed description when considered in connection with the accompanying drawings wherein:

FIG. 1A is an absorbance profile of a fractionation of cytoplasmic RNA on a sucrose density gradient wherein the absorbance (at 254 nm) is plotted against the sedimentation rate of the cytoplasmic RNA;

FIG. 1B is a photograph of purified RNA electrophoresed on an agarous gel and stained with ethidium bromide illustrating the fractionation of RNA;

FIG. 2 is a color representation of DNA chip hybridization results comparing probes of total RNA to probes derived from polysomal RNA (translational probes);

FIG. 3 is a color representation of DNA chip hybridization results comparing probes of total RNA (Tot) to probes derived from nuclear RNA (STP);

FIGS. 4A-C are schematic representations of plasmids that contain the Polio virus 2A genes (A) in plasmid pTK-OP3-WT2A, (B) in the plasmid miniTK-WT2A, and (C) in a plasmid containing a hygromycin selectable marker;

FIG. 5 is graph illustrating the induction of Polio virus 2A protease leading to cell death after induction of the 2A protease;

FIG. 6 is a photograph of a gel illustrating the presence of Polio virus 2A protease expression in transformed HEK-293 cells (293-2A) following induction with IPTG and the absence of the Polio virus 2A protease in HEK-293 (293) parental cells following treatment with IPTG; and

FIG. 7 is a photograph of a Western blot illustrating the activity of the Polio virus 2A protease in cleaving the p220 protein component of the 40S ribosomal subunit demonstrating that clones which were induced for Polio virus 2A protease generated cleavage products of the p220 protein.

### DETAILED DESCRIPTION OF THE INVENTION

A method of identifying genes whose expression is regulated at least in part at the mRNA level by selectively stimulating an unknown target mRNA with a stress inducing element, the target mRNA being part of a larger sample. The organism may be any organism which provides suitable mRNA. The mRNA sample is derived from cellular compartments based on expression regulation and protein localization which are differentially analyzed to identify genes which are translationally regulated by the stress inducing element. This method is designed for identifying and cloning genes which are responsive to specific cues. That is, the present method is designed for identifying and cloning genes which are either up- or down-regulated responsive to a specific pathology, stress, physiological condition, and so on, and in generally to any factor that can influence cells or organisms to alter their gene expression.

The method of the present invention provides a novel approach to the identification and cloning of genes that are involved in fundamental cellular functions and which are regulated at any level in an organism. The basic underlying theory for this method relies on the knowledge that the regulation of gene expression can be controlled at different levels (modes) and that each different regulation levels is manifested by some difference in the distribution of the specific mRNAs in the cell. In genes that are regulated by translation, the mRNA is stored in the cell in an inactive form and will not be found on polysomes. Following the appropriate external cue, the mRNA is incorporated into the polysomes and translated, and the encoded protein quickly appears. By comparing mRNA populations that are “active” or “non-active” at a given time, genes that are regulated by a mechanism referred to as the shift mechanism can be identified.

Genes whose main regulatory level is the active transport of mRNA from the nucleus to the cytoplasm are stored in the nucleus and at the appropriate cue the mRNA is transported to the cytoplasm. Comparison of mRNA isolated from the nucleus and cytoplasm before and after the cue can lead Lo the discovery of genes controlled in this way. The comparison of mRNA derived from the nucleus also allows direct analysis of the transcription activity of many genes. For most transcriptionally activated genes a basal level of mRNA exists in the cell even when the basal transcription activity is low. Thus, increased transcription (up to five-fold) is often obscured when total cellular RNA is used for differential analysis of gene expression. The use of nuclear RNA allows direct measurement of transcription activity of many genes, since the basal mRNA is found in the cytoplasm. The result is a major increase in sensitivity for the detection of differential expression.

In the case of mRNA stability regulation, it is expected that such mRNA would be similarly transcribed before and after cue administration, resulting in a similar abundance in nuclear mRNA pools. However, if the mRNA is stabilized following the cue, its abundance in the cytoplasm would become higher. In the case of mRNA transport regulation, such mRNA is expected to exist at a high level in the nucleus and a low level in the cytoplasm prior to the cue, which situation would be reversed after administration of the cue. It is thus easy to differentiate between the two regulatory modes.

The method of the invention includes the identification of genes regulated at the translational level; genes regulated at the transcription level; genes regulated by RNA stability; genes regulated by mRNA transport rate between the nucleus and the cytoplasm; and genes regulated by differential splicing. That is, genes whose expression is at least partly controlled or regulated at the mRNA level can be identified.

The method will identify genes encoding secreted and membrane proteins; genes encoding for nuclear proteins; genes encoding for mitochondrial proteins; and genes encoding for cytoskeletal proteins. In addition, any other gene whose expression can be controlled at the mRNA level can be identified by this method.

As used herein, RNA refers to RNA isolated from cell cultures, cultured tissues or cells or tissues isolated from organisms which are stimulated, differentiated, exposed to a chemical compound, are infected with a pathogen or or otherwise stimulated. As used herein, translation is defined as the synthesis of protein on an mRNA template.

As used herein, stimulation of translation, transcription, stability or transportation of unknown target mRNA or stimulating element, includes chemically, pathogenically, physically, or otherwise inducing or repressing an mRNA population from genes which can be derived from native tissues and/or cells under pathological and/or stress conditions. In other words, stimulating the expression of a genes mRNA with a stress inducing element or “stressor” can include the application of an external cue, stimulus, or stimuli which stimulates or initiates translation of a mRNA stored as untranslated mRNA in the cells from the sample. The stressor may cause an increase in stability of certain mRNAs, or induce the transport of specific mRNAs from the nucleus to the cytoplasm. The stressor may also induce gene transcription. In addition to stimulating translation of mRNA from genes in native cells/tissues, stimulation can include induction and/or repression of genes under pathological and/or stress conditions. The present method utilizes a stimulus or stressor to identify unknown target genes which are regulated at the various possible levels by the stress inducing element or stressor.

The method of the present invention synergistically integrates two types of previously known methodologies which were otherwise used separately. The first method is the division of cellular mRNA into separate pools of mRNA derived from polysomes, nucleus, cytoplasm or spliceosomes. The second methodology involves the simultaneous comparison of the relative abundance of the mRNA species found in the separate pools by a method of differential analysis such as differential display, representational difference analysis (RDA), gene expression microarray (GEM), suppressive subtraction hybridization (SSH) Diatchenko et al., 1996), and oligonucleotide chip techniques such as the chip technology exemplified by U.S. Pat. No. 5,545,531 to Rava et al. assigned to Affymax Technologies N.V. and direct sequencing exemplified by WO 96/17957 patent application to Hyseq, Inc.

Briefly, subtractive hybridization is defined as subtraction of mRNA by hybridization in solution. RNAs that are common to the two pools form a duplex that can be removed, enriching for RNAs that are unique or more abundant in one pool. Differential Display is defined as reverse transcription of mRNA into cDNA and PCR amplification with degenerated primers. Comparison of the amounts amplification products (by electrophoresis) from two pools indicate transcript abundance. RDA, GEM, SSH, SAGE are described herein above.

The specific cells/tissues which are to be analyzed in order to identify translationally regulated genes, can include any suitable cells and/or tissues. Any cell type or tissue can be used, whether an established cell line or culture or whether directly isolated from an exposed organism.

The cells/tissues to be analyzed under the present method are selectively stimulated or “stressed” utilizing a physiological, chemical, environmental and/or pathological stress inducing element or stressor, in order to stimulate the translation of ETA within the sample tissue and identify genes whose expression is regulated at least in part at the mRNA level. Stimulation can cause up or down regulation. Following stimulation, RNA is isolated or extracted from the cells/tissues. The isolation of the RNA can be performed utilizing techniques which are well known to those skilled in the art and are described, for example, in “Molecular Cloning; A Laboratory Manual” (Cold Springs Harbor Laboratory Press, Cold Spring Harbor, N.Y. 1989). Other methods for the isolation and extraction of RNA from cells/tissue can be used and will be known to those of ordinary skill in the art. (Mach et al., 1986, Jefferies et al., 1994). However, may variations of these methodologies have been published. The methods described herein were carefully selected after many trials.

The mRNAs which are actively engaged in translation and those which remain untranslated can be separated utilizing a procedure such as fractionation on a sucrose density gradient, high performance gel filtration chromatography, or polyacrylamide gel matrix separation (Ogishima et al., 1984, Menaker et al., 1974, Hirama et al., 1986, Mechler, 1987, and Bharucha and Murthy, 1992), since mRNAs that are being translated are loaded with ribosomes and, therefore, will migrate differently on a density gradient than ribosome-free untranslated mRNAs. By comparing mRNA populations that are active or non-active in translation at a given time, genes that are regulated by the “shift mechanism” can be identified.

Polysomal fractionation and specific analysis can be facilitated by treatment of target cell/tissue with drugs that will specifically inhibit or modulate transcription or translation. Examples of such drugs are actinomycin D and cyclohexamide, respectively.

The fractionation can be completed to create polysomal subdivisions. The subdivisions can be made to discriminate between total polyribosomes or membrane bound ribosomes by methods known in the art Mechler, 1987). Further, the mRNA sample can additionally be fractionated into one or more of at least the following subsegments or fractions: cytoplasmatic, to nuclear, polyribosomal, sub polyribosomal, microsomal or rough endoplasmic reticulum, mitochondrial and splicesome associated mRNA by methods known in the art (see also Table 1).

More specifically, nuclear fractions can be obtained using the method set forth in the article entitled Abundant Nuclear Ribonucleoprotein Form of CAD RNA (Sperlang, 1984) as set forth in the Experimental section, thus allowing nuclear RNA to be utilized for a method of identifying genes which are regulated or responsive to stress conditions. Further, antisense RNA can be utilized as a method for identifying genes which are responsive to specific pathology or stress conditions. Antisense RNA can be isolated using the methods described by Dimitrijevic, whose abstract details the methods utilized for obtaining and isolating antisense RNA from a sample. Additionally, microsomal fractions may be obtained using the methods of the present invention as set forth in the Experimental Section which are modifications of the methods disclosed by Walter and Blobel in 1983.

Following isolation and division of the total mRNA population into separate expression regulation and protein localization pools of mRNA, the relative abundance of the many mRNA species found in these pools are simultaneously compared using a differential analysis technique such as differential display, oligonucleotide chips, representational difference analysis (RDA) GEM-Gene Expression Microarays (Schena et al., 1995, Aiello et al., 1994, Shen et al., 1995, Bauer et al., 1993, Liang and Pardee, 1992, Liang and Pardee, 1995, Liang et al., 1993, Braun et al., 1995, Hubank and Schatz, 1994) and suppressive subtraction hybridization (SSH). The RNA isolated from the fractions can be further purified into mRNA without the ribosomal RNA by poly A selection. It should be noted that multiple pools can be analyzed utilizing this method. That is, different cell aliquots subjected to different stressors can be compared with each other as well as with the reference sample.

Labeled nucleic acid probes (in a cDNA ,PCR product or rRNA transcribed from the cDNA) made from RNA derived from polysomal, non-polysomal, mRNPs, nuclear, cytoplasmic, or spliceosome fractions can be used as probes, to identify clones of cDNA, genomic clones, and mRNA species that are fixed onto a solid matrix-like microarrays such as (GEM), that shown in U.S. Pat. No. 5,545,531 to Rava et al. and WO96/17957 to Hyseq, Inc., and membranes of any kind where clones can be either blotted after electrophoresis or directly loaded (dot blot) onto the membrane. The label can be radioactive, fluorescent, or incorporating a modified base such as digoxigenin and biotin.

Comparison between the fractions derived from the polysomal or polyribosomal fraction or other fractions to the total unfractionated material is essential to discriminate between differentials in expression levels that are the result of transcription modulation from those that result from modulation of translation per se. The polysomal fractions or groups can include membrane bound polysomes, loose or tight polysomes, or free unbound polysome groups.

The importance of utilizing the polysomal sub-population in order to identify differentially (translationally) expressed genes is shown in Example 1 where a number of genes were not detected as translationally expressed under heat shock inducement when total mRNA was used as the detection probe but, however, when polysomal mRNA was used as a probe, a number of genes were identified as differentially expressed. As shown in Example 1, a number of genes under heat shock inducement with total mRNA derived probe were detected when probed with polysomal mRNA fractions. Heat shock, being a model for acute diseases such as ischemic diseases, reveal the importance of the polysomal probe. Cells store critical mRNAs in an inactive form so that in an acute situation they can be quickly loaded onto polysomes (without the need to wait for their production by transcription) and translated to produce the proteins the cells require for their survival under stress.

The present method for identifying translationally regulated genes is not limited by the source of the mRNA pools. Therefore, the present method can be utilized to clone genes from native cells/tissue under pathological and/or stress conditions that are regulated by the “shift mechanism,” as well as genes that are induced/repressed under pathological and/or stress conditions. Pathologies can include disease states including those diseases caused by pathogens and trauma Stress conditions can also include disease states, physical and psychological trauma, and environmental stresses. Following analysis by the selected method of differential analysis, the genes which have been identified as being regulated by translation can be cloned by any suitable cloning methodologies known to those skilled in the art (Lisitsyn and Wigler, 1993).

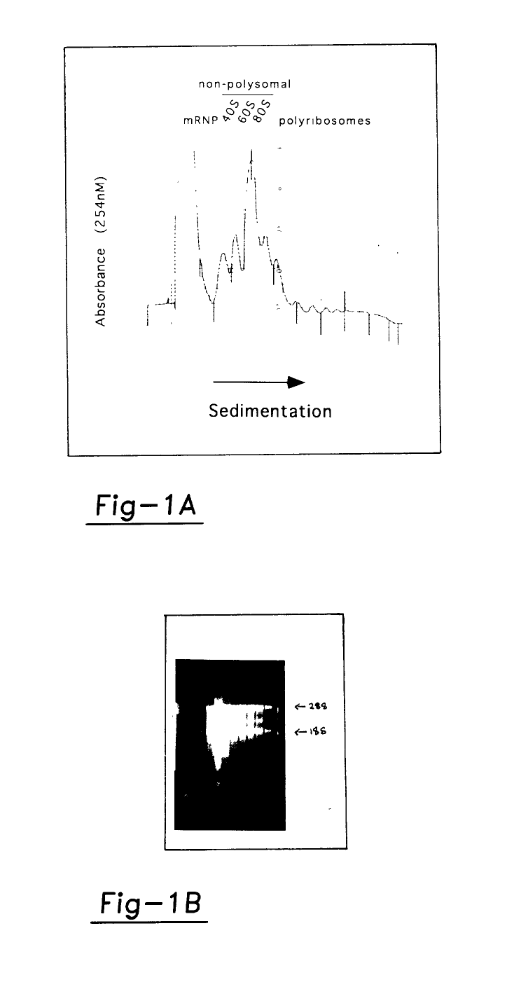

Differential comparisons can be made of all possible permutations of polysomal vs. non-polysomal RNA where the definition of the fraction type is done, for example, by absorbance profile at 254 nm, density of the sucrose gradient as shown in FIG. 1A (or another size standard if high pressure liquid chromatography or gel systems are used) and types of RNA that are stained with ethidium bromide after electrophoresis of the fractions on agarous gels are completed, as shown in FIG. 1B. In FIG. 1A, the polysomal fractions are those that have mRNA with more than two ribosomes loaded. The materials and methods for this comparison are set forth below in the experimental section.

Differential comparisons can also include polysomal vs. non-polysomal fractions in each condition By “condition” it is meant that cells from the same source, such as a cell line, a primary cell, or a tissue that undergoes different treatment or has been modified to have different features or to express different sets of genes. For example, this can be accomplished by differentiation, transformation, application of the stress such as oxygen deprivation, chemical treatment, or radiation. Permutations can include, for example:

1. polysomal fractions between conditions individually (migrating in the same density) or in a pool;

2. non-polysomal fractions between conditions individually (migrating in the same density) or in a pool;

3. non-polysomal to polysomal between conditions and within each condition individually (migrating in the same density) or in a pool; and

4. each of the fractions being polysomal and non-polysomal individually (migrating in the same density) or in a pool that can be compared to total RNA that is unfractionated.

The method described above for the identification of genes regulated on the translational level has a number of applications. A particular application for this method is its use for the detection of changes in the pattern of mRNA expression in cells/tissue associated with any physiological or pathological change. By comparing the translated versus untranslated mRNAs, the effect of the physiological or pathological cue or stress on the change of the pattern of mRNA expression in the cell/tissue can be observed and/or detected. This method can be used to study the effects of a number of cues, stimuli, or stressors to ascertain their effect or contribution to various physiological and pathological activities of the cell/tissue. In particular, the present method can be used to analyze the results of the administrations of pharmaceuticals (drugs) or other chemicals to an individual by comparing the mRNA pattern of a tissue before and after the administration of the drug or chemical. This analysis allows for the identification of drugs, chemicals, or other stimuli which affect cells/tissue at the level of translational regulation. Utilizing this method, it is possible to ascertain if particular mRNA species are involved in particular physiological or disease states and, in particular, to ascertain the specific cells/tissue wherein the external stimulus, i.e., a drug, affects a gene which is regulated at the translational level.

The identification of a subgroup of genes regulated on the translational level involved a method for identifying gene sequences coding for internal ribosome entry sites (IRES), including the general steps of inhibiting 5′cap-dependant mRNA translation in a cell, collecting a pool of mRNA from the cells, and differentially analyzing the pool of mRNA to identify genes with sequences coding for internal ribosome entry sites.

As described above, it is known that an exception to the standard 5′-cap dependent translation initiation exists. Sequences exist within untranslated regions (UTRs) of RNAs which can include the presence of specific sequences known as internal ribosome entry sites (IRES). (Ehrenfeld, 1996) These internal ribosome entry sites have been shown to support translation initiation for several prokaryotic and eukaryotic systems as set forth above. However, in order to identify translationally controlled genes via 5′-cap independent translation to mechanisms and their association with both normal and abnormal processes, it is necessary to inhibit 5′-cap initiated translation so that 5′-cap independent mRNA translation can be selected for. This inhibition is necessary since IRES sequences are difficult, if not impossible, to identify by sequence homology.

In order to inhibit 5′-cap dependent translation and thereby select for the presence of 5′-cap independent translation, cells or tissues which are to be analyzed for the presence of internal ribosome entry sites must be treated in some manner to prevent or discourage the 5′-cap translation initiation mechanism. The mechanism(s) of standard scanning-type translation initiation should be substantially, if not totally, turned off or shut down to, in essence, shift the translation equilibrium in favor of IRES initiated translation. That is, recognition of the 5′-cap structure is inhibited by disrupting the normal mechanism for 5′-cap mediated initiation. The mechanism for inhibiting the 5′-cap translation can include any known means or mechanisms for preventing the initiation of 5′-cap mediated translation. One such mechanism for inhibiting 5′-cap mediated translation is the expression of Polio virus 2A protease into a cell, cell system, or tissue to be analyzed for the presence of IRES sequences. The use of the Polio virus 2A protease inhibits 5′-cap-dependent mRNA translation by inactivating the cellular 5′-cap-dependent translation machinery. This enables the identification of cellular IRES containing genes which may be transitionally controlled and play a critical role in the immediate response of the cell following the application of a stress inducing element/stressor such as heat shock, hypoxia, or other stress inducing elements as set forth above, prior to gene activation. The Polio virus 2A protease prevents 5′-cap-mediated translation by cleaving the large sub-unit of eIF-4γ (p220) of eukaryotic translation initiation factor 4 (eIF-4) which is involved in the recognition of the mRNA 5′-cap.

In order to inhibit the 5′-cap-mediated translation, the Polio virus 2A protease must be incorporated into the cell or cells being analyzed for the presence of gene sequences coding for internal ribosome entry sites and/or for identifying translationally regulated genes. One such method for incorporating the Polio virus 2A protease into a cell involves the transformation of a target cell with an expression vector containing the gene which codes for the Polio virus 2A protease. Because the Polio virus 2A protease is deleterious to living cells when it is constitutively expressed, the expression vector containing the Polio virus 2A protease gene is coupled with a bacterial LacI inducible system wherein a LacI repressor is constitutively expressed under a CMV promoter. The Polio virus 2A is protease may be expressed under a number of suitable promoters including the RSV, the TK, or the mini-TK promoter coupled at their 3′ end to the LacI repressor binding sites. By transforming the target cells with an expression vector containing the LacI repressor and the Polio virus 2A expression vector, the expression of the Polio virus 2A protease can be induced upon treatment of the cells with isopropyl-β-D-thiogalatopyranoside (IPTG). Treatment of the target cells with IPTG relieves the binding of the Lad repressor molecules bound at the repressor binding sites thus enabling transcription of the Polio virus 2A protease. By coupling the expression of the Polio virus 2A protease to an inducible system, such as the LacI system, this mechanism allows for the establishment of control of the expression of the gene coding for the Polio virus 2A protease.

Following induction of the expression of the Polio virus 2A protease in the target cells, RNA, presumably containing internal ribosome entry sites, can be collected and analyzed utilizing the methods described above to identify genes whose translation is up-regulated by the effects of the Polio virus 2A protease.

### EXPERIMENTAL

### DIFFERENTIAL TRANSLATION

Materials and Methods

General Scheme

a. Total mRNA organic extraction of all RNA from the source tissue or cell. (additional selection for polyA+mRNA can be included).

b. Nuclear RNA-lysis of cells (from a tissue or a cell line) by homogenization in hypotonic buffer. Collection of nuclei by centrifugation and organic extraction of the RNA.

c. Cytoplasmic RNA—Organic extraction of the RNA from the supernatant from b above.

d. Polyribosomal/subpolyribosomal fractionation. Lysis of cells by homogenization hypotonic buffer removal of nuclei and fractionation of polyribosome on linear sucrose gradients and organic extraction of the RNA from each fraction of the gradient.

e. Secreted and membrane encoding transcripts.

1. Isolation of RER on Percol gradients (after homogenization of cells).

2. Preparation of microsomes containing the RER

3. Isolation of membrane-bound polyribosomes by successive treatment of cells with detergents.

f. Nuclear proteins. Isolation of cytoskeletal associated polyribosomes by treating cells lyzates with different detergents.

g. Mitochondrial genes. Isolation of mitochondria on Percoll gradients.

h. Alternative splicing. Separation of nuclei and isolation of splicsosome (proteins and RNA complex) on linear sucrose gradients.

Preparation of cell extracts

Cells were centrifuged. The pellet was washed with PBS and recentrifuged. The cells were resuspended in 4× of one packed cell volume (PCV) with hypotonic lysis buffer (HLB: 20mM TrisHCL pH=7.4; 10mM NaCl; 3mM MgCl₂). The cells were incubated five minutes on ice. 1×PCV of HLB containing 1.2% Triton X-100 and 0.2 M sucrose was added. The cells were homogenized with a Dounce homogenizer (five strokes with B pestle). The cell lysate was centrifuged at 2300 g for ten minutes at 4° C. The supernatant was transferred to a new tube. HLB containing 10 mg/ml heparin was added to a final concentration of 1 mg/ml heparin. NaCl was added to a final concentration of 0.15M. The supernatant was frozen at −70° C. after quick freezing in liquid N₂ or used immediately.

Sucrose gradient fractionation

A linear sucrose gradient from 0.5 M to 1.5 M sucrose in HLB was prepared. Polyallomer tubes (14×89 mm) were used. 0.5 to 1.0 ml of cell extract was loaded on the gradient. The cells were centifuged at 36,000 RPM for 110 minutes at 4° C. An ISCO Density Fractionator was used to collect the fractions and record the absorbance profile.

RNA purification

SDS was added to 0.5% and Proteinase K to 0.1mg/ml and incubated at 37° C. for 30 minutes. Extract with an equal volume of phenol+chloroform (1:1). The aqueous phase was extracted with one volume of chloroform and the RNA was precipitated by adding Na-Acetate to 0.3 M and 2.5 volumes of ethanol and incubating at −20° C. overnight. Centrifuged ten minutes, the supernatant was aspirated and the RNA pellet was dissolved in stere, diethylpyrocarbonate (hereinafer referred to as “DEPC”) DEPC-treated water.

Preparation of Microsomes

When possible fresh tissues and cells are used, without freezing. Tissues were powdered in liquid nitrogen with mortar and pestle and then homogenized using 4 ml of buffer A/1 gr tissue (Buffer A is 250mM sucrose, 50 M TEA, 50mM KOAc pH 7.5, 6mM Mg(Oac)₂, 1mM EDTA, 1mM DTT, 0.5mM PMSF. PMSF was made in ethanol before making the buffer and added in drops to buffer while being stirred. This was stirred for 15 minutes and then DTT was added). Fresh organs were washed in Buffer A a few times, and then cut into pieces and homogenized. Approximately 5 ml buffer A/5×10⁸ a cells were added and homogenized. This was then homogenized on ice for 5-10 times, or as needed with the individual tissue. The mixture was transferred to 50 ml tubes, then centrifuged for 10 minutes, at 4° C. in a swinging bucket rotor machine. Next, the supernatant was transferred, avoiding the pellet as much as possible, to a Sorvall tube, the pellet was washed again with 1 ml buffer and centrifuge as before. The two pellets were combined, us establishing the nuclear fraction. The combination was dissolved and treated the pellet with Tri-reagent (usually 2 ml of Tri-reagent when sample is from cells) to extract the nuclear RNA. The combined 1st and 2nd supernatants were centrifuged for 10 minutes at 10000 g at 4° C. Again, the supernatant was transferred to a tube and kept on ice. The pellet was washed again with 1 ml buffer and centifuged for 10 minutes at 10000 g and the two pellets were combined as before, thus establishing the Mitochondrial pellet. Again, the pellet was treated with Tri-reagent (usually 1 ml with cells) and the Mitochondrial RNA is was extracted. Next, cold ultracentrifuge tubes were prepared containing a sucrose cushion made of: buffer A+1.3 M sucrose. The volume of the cushion was approximately 1/3 of the supernatant. The supernatant was loaded on the cushion in a 1:3 ratio of cushion to supernatant. A pair of tubes was weighed for balancing, a 20-30 mg difference is allowable. The tubes were centrifuged 2.5 hours at 140,000 g, 4° C. with a Ti60.2 rotor (45,000 rpm). When two phases of supernatant were visible, then the red phase only was transferred (if possible), as the cytoplasmic fraction, to a sorvall tube. The clear supernatant was aspirated. When not possible to separate or phase distinction not visible, all the supernatant was taken as cytoplasmic fraction and dilute sucrose with TE (10mM Tris-HCl pH 8.0, 1 ml EDTA). In the pellet were the microsomes which were visible and were clear or yellowish. For the RNA extraction, the cytoplasmic fraction was treated with 1% SDS, 0.1mg/ml proteinase K, for 30 minutes, at 37° C. After this, freezing at −80° C. was possible. The RNA was extracted with a phenol:chloroform combination and precipitate with 0.3 M Na-acetate, 1 μl glycogen, and equal volume of isopropanol. O'N precipitation was possible and can be accomplished at 30 minutes on ice. The extract was spun at 10000g, for 20 minutes, then the RNA pellet was washed with 70% ethanol. The pellet was dried and then dissolved in H₂O. The microsomes were then dissolved with 0.1 M NaCl/1% SDS solution (1ml is usually sufficient for a small pellet) and extracted with a phenol:chloroform combination (no proteinase K treatment). Then the precipitation of the RNA was done in the same way as for the cytoplasmic fraction but without the requirement of adding salt.

Preparation of Nuclear and Cytoplasmic RNA

Subconfluent plates were washed with 125 mM KCl-30 mM Tris-hydrochloride (pH 7.5)−5 mM magnesium acetate-1 mM 2-mercaptoethanol-2 mM ribonucleoside vanadyl complex (2)-0.15 mM spermine-0.05 mM spermidine at 4° C., and cells scraped from the plates were washed twice with the same buffer. Approximately 10⁸ cells were allowed to swell for 10 minutes in 2.5 ml of swelling buffer (same as wash buffer except the KCl concentration was 10 mM) lysed with 20 strokes of a Dounce homogenizer (B pestle), overlaid on an equal volume of swelling buffer containing 25% glycerol, and centrifuged for 5 min. at 400×g and 4° C. The upper layer of the supernatant, which contained 90% of the CAD sequences released by lysis, was designated the cytoplasmic fraction. The nuclear pellet was washed once with 2 ml of swelling buffer-25% glycerol-0.5% Triton X-100 and once with 2 ml of swelling buffer.

Nuclear RNP. Nuclei from 10⁸ cells, prepared as described above, were suspended in 1 ml of 10 mM Tris-hydrochloride (pH 8.0)-100 mM NaCl-2 mM MgCl₂-1 mM 2-mercapthoethanol-0.15 mM spermine-0.05 mM spermidine-10 mM ribonucleoside vanadyl complex (2)-100 U of placental RNase inhibitor (Amersham Corp.) per ml and sonicated at the maximum power setting of a Konres micro-ultrasonic cell disrupter for 20 g at 4° C. Bacterial tRNA (2 mg) was added, to adsorb basic proteins (9), and the mixture was centrifuged for 1 minute (Eppendorf microcentrifuge). The supernatant was applied to a 15 to 45% sucrose gradient in mM Tris-hydrochloride-100 mM NaCl-2 mM MgCl₂-2 mM ribonucleoside vanadyl complex and centrifuged in a Beckman SW41 rotor for 90 minutes at 40,000 rpm and 4° C. RNA was recovered from gradient fractions by the addition of sodium dodecyl sulfate to 0.5%, treatment with proteinase K (200 μg/ml) for 2 hours at 37° C., extraction with phenol, and precipitation with ethanol.

Preparation of Antisense RNA

Total cellular RNA is extracted. Part of the RNA pool is immobilized on a membrane, another part converted into cDNA after ligation of oligodeoxynucletides to the 3′-ends. The use of biotinylated, complementary oligos for cDNA synthesis allows immobilization of a “minus” strand to streptavidin-coated magnetic beads. A second set of oligos is ligated to the cDNA at the previous 5′-end of the RNA. Plus strands are eluted from the bound strands and hybridized to the membrane-bound RNA. Since the cDNA strand used has the same polarity of the RNAs, only cDNA sequences that can bind to complementary RNAs should be retained. PCR amplification and subsequent cloning of PCR-fragments is followed by sequence analysis. To test whether cloned sequences are correctly identified, probes are generated in sense and antisense direction. Positive clones will be structurally and functionally characterized. In order to work out this method, we started using a bacterial strain (Escherichia coli), containing plasmid R1 that regulates its copy number by antisense RNA. Previous work has identified both antisense (CopA) and target RNA (CopT) of R1 intracellularly. This procedure, if feasible, will then be used to screen for antisense RNA systems in other organisms.

DIFFERENTIAL ANALYSIS

Differential display:

Reverse transcription: 2 μg of RNA were annealed with 1pmol of oligo dT primer (dT)₁₈ in a volume of 6.5 μl by heating to 70° C. for five minutes and cooling on ice. 2 μl reaction buffer (×5), 1 μl of 10mM dNTP mix, and 0.5 μl of SuperScript II reverse transcriptase (GibcoBRL) was added. The reaction was carried out for one hour at 42° C. The reaction was stopped by adding 70 μl TE (10mM Tris pH=8; 0.1mM EDTA). Oligonucleotides used for Differential display: The oligonucleotides were essentially those described in the Delta RNA Fingerprinting kit (Clonetech Labs. Inc.). There were 9 “T” oligonucleotides of the structure: 5′ CATTATGCTGAGTGATATCTTTTTTTTTXY 3′(SEQ ID No: 1). The 10 “p” oligonucleotides were of the structure: 3′ATTAACCCTCACTAAA “TGCTGGGGA” 3′(SEQ ID No: 11) where the 9 or 10 nucleotides between the parenthesis represent an arbitrary sequence and there are 10 different sequences (SEQ ID Nos. 12-21), one for each “P” oligo.

Amplification reactions: each reaction is done in 20 μl and contains 50 μM dNTP mix, 1 μM from each primer, 1× polymerase buffer, 1 unit expand Polymerase (Beohringer Mannheim), 2 μCi [α−³²P]dATP and 1 μl cDNA template. Cycling conditions were: three minutes at 95° C., then three cycles of two minutes at 94° C., five minutes at 40° C., five minutes at 68° C. This was followed by 27 cycles of one minute at 94° C., two minutes at 60° C., two minutes at 68° C. Reactions were terminated by a seven minute incubation at 68° C. and addition of 20g1 sequencing stop solution (95% formamide, 10nM NaOH, 0.025% bromophenol blue, 0.025% xylene cyanol).

Gel analysis: 3-4 μl were loaded onto a 5% sequencing polyacrylamide gel and samples were electrophoresed at 2000 volts/40 milliamperes until the slow dye (xylene cyanol) was about 2 cm foam the bottom. The gel was transferred to a filter paper, dried under vacuum and exposed to x-ray film.

Recovery of differential bands: bands showing any a differential between the various pools were excised out of the dried gel and placed in a microcentrifuge tube. 50 μl of sterile H₂O were added and the tubes heated to 100° C. for five minutes. 1 μl was added to a 49 μl PCR reaction using the same primers used for the differential display and the samples were amplified for 30 cycles of: one minute at 94° C., one minute at 60° C. and one minute at 68° C. 10 μl was analyzed on agarous gel to visualize and confirm successful amplification.

### REPRESENTATIONAL DIFFERENCE ANALYSIS

Reverse transcription: as above but with 2 μg polyA+selected mRNA. Preparation of double stranded cDNA: cDNA from previous step was treated with alkali to remove the mRNA, precipitated and dissolved in 20 μl H₂O. 5 μl buffer, 2 μl 10mM dATP, H₂O to 48 μl and 2 μl terminal deoxynucleotide transferase (TdT) were added. The reaction was incubated 2-4 hours at 37C. 5 μl oligo dT (1 μg/μ1) was added and incubated at 60° C. for 5 minutes. 5 μl 200 mM DTT, 10 μl 10× section buffer (100 mM Mg Cl₂, 900 mM Hepes, pH 6.6) 16 μl dNTPs (1 mM), and 16 U of Klenow were added and the mixture was incubated overnight at room temperature to generate ds cDNA. 100 μl TE was added and extracted with phenol/chloroform. The DNA was precipitated and dissolved in 50 μl H₂O.

Generation of representations: cDNA with DpnlI was digested by adding 3 μl DpnlI reaction buffer 20 V and DpnlI to 25 μl cDNA and incubated five hours at 37° C. 50 μl TE was added and extracted with phenol/chloroform. cDNA was precipitated and dissolved to a concentration of 10 ng/μl.

The following oligonucleotides are used in this procedure:

R-Bgl-12 5′ GATCTGCGGTGA 3′(SEQ ID No: 22)

R-Bgl-24 5′ AGCACTCTCCAGCCTCTCACCGCA 3′(SEQ ID No: 23)

J-Bgl-12 5′ GATCTGTTCATG 3′(SEQ I No: 24)

J-Bgl-24 5′ ACCGACGTCGACTATCCATGAACA 3′(SEQ ID No: 25)

N-Bgl-12 5′ GATCTTCCCTCG 3′(SEQ ID No: 26)

N-Bgl-24 5′ AGGCAACTGTGCTLCCGAGGGAA 3′(SEQ ID No: 27)

R-Bgl-12 and R-Bgl-24 oligos were ligated to Tester and Driver: 1.2 μg DpnII digested cDNA. 4 μl from each oligo and 5 μl ligation buffer X10 and annealed at 60° C. for ten minutes. 2 μl ligase was added and incubated overnight at 16° C. The ligation mixture was diluted by adding 140 μl TE. Amplification was carried out in a volume of 200 μl using R-Bgl-24 primer and 2 μl ligation product and repeated in twenty tubes for each sample. Before adding Taq DNA polymerase, the tubes were heated to 72° C. for three minutes. PCR conditions were as follows: five minutes at 72° C., went cycles of one minute at 95° C. and three minutes at 72° C., followed by ten minutes at 72° C. Every four reactions were combined, extracted with phenol/chloroform and precipitated. Amplified DNA was dissolved to a concentration of 0.5 μg/μl and all samples were pooled.

Subtraction: Tester DNA (20kg) was digested with DpnlI as above and separated on a 1.2% agarous gel. The DNA was extracted from the gel and 2 μg was ligated to J-Bgl-12 and J-Bgl124 oligos as described above for the R-oligos. The ligated Tester DNA was diluted to 10 ng/μl with TE. Driver DNA was digested with DpnII and repurified to a final concentration of 0.5 μg/μl. Mix 40 μg of Driver DNA with 0.4 μg of Tester DNA. Extraction was carried out with phenol/chloroform and precipitated using two washes with 70% ethanol, resuspended DNA in 4 μl of 30mM EPPS pH=8.0, 3mM EDTA and overlayed with 35 μl mineral oil. Denatured at 98° C. for five minutes, cool to 67° C. and 1 μl of 5 M NaCl was added to the DNA. Incubated at 67° C. for twenty hours. Diluted DNA by adding 400 μl TE.

Amplification: Amplification of subtracted DNA in a final volume of 200 μl as follows: Buffer, nucleotides and 20 μl of the diluted DNA were added, heated to 72° C., and Taq DNA polymerase was added. Incubated at 72° C. for five minutes and added J-Bgl-24 oligo. Ten cycles of one minute at 95° C., three minutes at 70° C. were performed. Incubated ten minutes at 72° C. The amplification was repeated in four separate tubes. The amplified DNA was extracted with phenol/chloroform, precipitated and all four tubes were combined in 40 μl 0.2XTE, Digested with Mung Bean Nuclease as follows: To 20 μl DINA 4 μl buffer, 14 μl H₂O and 2 μl Mung Bean Nuclease (10 units/μl ) was added. Incubated at 30° C. for thirty-five minutes+First Differential Product (DPI).

Repeat subtraction hybridization and PCR amplification at driver: differential ratio 1:400(DPII) and 1:40,000 (DPIII) using N-Bgl oligonucleotides and J-Bgl oligonucieotides, respectively. Differential products were cloned into a Bluescript vector at the BAM HI site for analysis of the individual clones.

### EXAMPLES

### Example 1

Analysis of Genes Regulated at a Translational Level in a Representative Heat Shock GEM Differential Expression System

Materials and Methods

The experimental cells were grown under both normal temperature (37° C.) and heat shock temperature (43° C) for four hours. The cells were then harvested and cytoplasmic extracts were obtained, polysomes were fractionated and RNA extracted therefrom. From parallel cultures of cells, total cellular RNA was extractedThen, the extracted RNA was analyzed utilizing GEM technology as disclosed above.

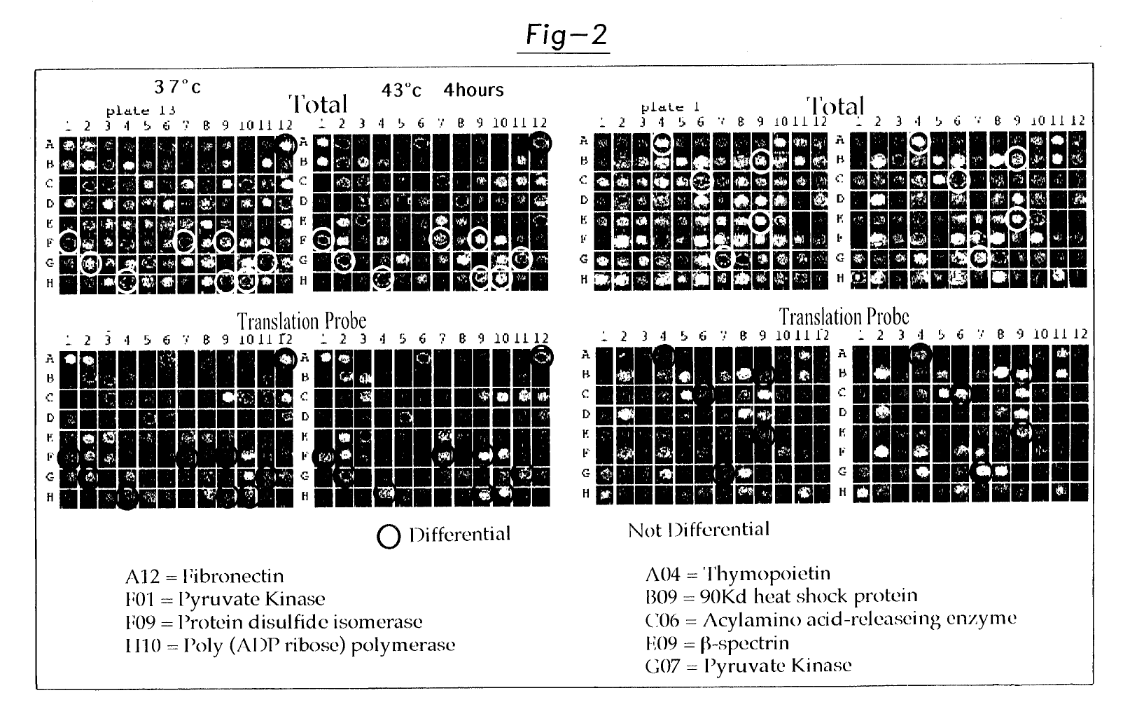

FIG. 2 and Tables 2 and 3 demonstrate the utility of utilizing polysomal probes versus total mRNA probes in differential expression analysis to identify genes which are differentially expressed in response to a stimulus such as heat shock. These Tables illustrate that fibronectin, pyruvate kinase, protein disulfide isomerese, poly(ADPribose) polymerase, thymopoietin, 90Kd heat shock protein, acylamino acid-releasing enzyme, β-spectrin, and pyruvate kinase were all identified as being differentially expressed utilizing a polysomal probe whereas, with the exception of fibronectin, the other proteins were not identified as being differentially expressed when a total mRNA probe was utilized. This example demonstrates the utility of the present invention for identifying translationally or differentially regulated genes which are regulated by a stress inducing element. Additionally, in Table 2, the results of heat shock differential gene expression analysis with both polysomal probes and total mRNA probes is provided. Table 2 illustrates that a number of differentially expressed genes were identified using a polysomal probe whereas when a total mRNA probe was used, these genes were not necessarily identified as being differentially expressed. Table 3 statistically illustrates the number of differentially expressed genes identified utilizing either total mRNA or polysomal mRNA as a probe. Table 3 clearly illustrates that polysomal mRNA probes yielded between two and greater than ten fold increases in the number of differentially expressed genes versus total mRNA probes.

### Example 2

Analysis of Genes at a Transcriptional Level using Nuclear mRNA Probes

The experimental cells were grown alternatively under normal conditions, for 4 hours under hypoxia (<1% oxygen) and for 16 hours under hypoxia. The cells were harvested and RNA was extracted either from nuclei that were prepared from the cells (nuclear RNA) or from extracts of unfractionated cells (total cellular RNA).

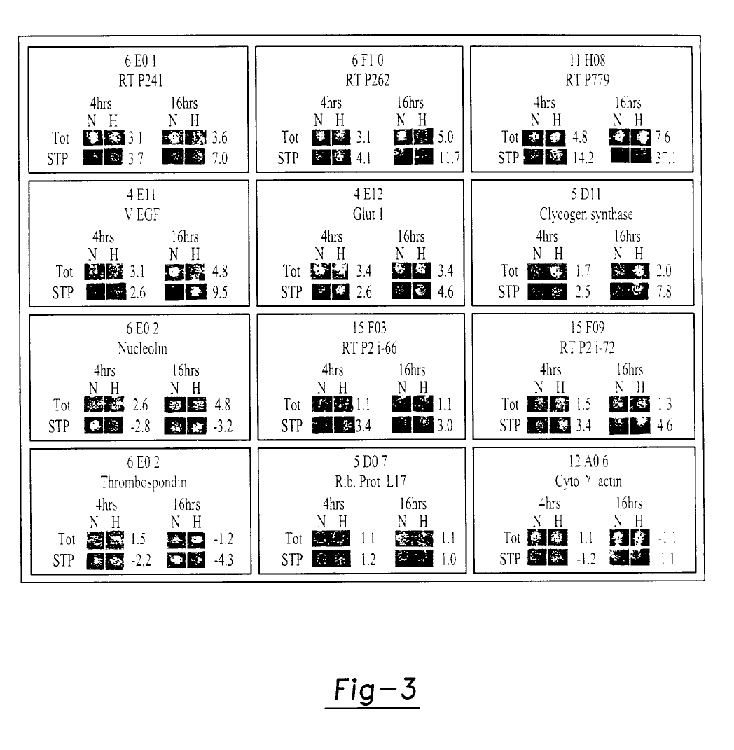

FIG. 3 demonstrates how the probes prepared from the nuclear RNA (STP) give a higher differential expression than the total cellular RENA probe (Tot). The control genes encoding VEGF (vascular endothelial growth factor), Glut1 (glucose transporter 1) and glycogen synthase are known to be induced by the hypoxia stress. The level of induction observed in the nuclear probe is much higher Man that seen in the total probe and much closer to the actual know level of induction. The three new genes RTP 241, RTP 262 and RTP 779 show marked induction by hypoxia. Again, the induction level seen with the nuclear probe is much higher, up to fivefold higher, as seen for RTP 779. When the induction of these genes was analyzed by the Northern blot method, it was found that the nuclear probe was once again much closer to the actual situation, while the total probe gives a marked underestimation.

The genes RTPi-66 and RTP21-72 demonstrate the ability of the nuclear probe to detect differentially expressed genes that do not appear differentially with the total probe.

The genes for Nucleolin and Thrombospondin show that also for down-regulated mRNAs the nuclear probe is much more sensitive and gives much high levels of differential expression values.

Lastly, The genes for ribosomal protein L17 and cytoplasmic gamma-actin are know as genes that do not respond to hypoxia stress. The nuclear probe and the total probe both show that no induction occurs.

### Example 3

Identification of IRES Containing Genes

Establishment of mammalian cells expressing 2A protease

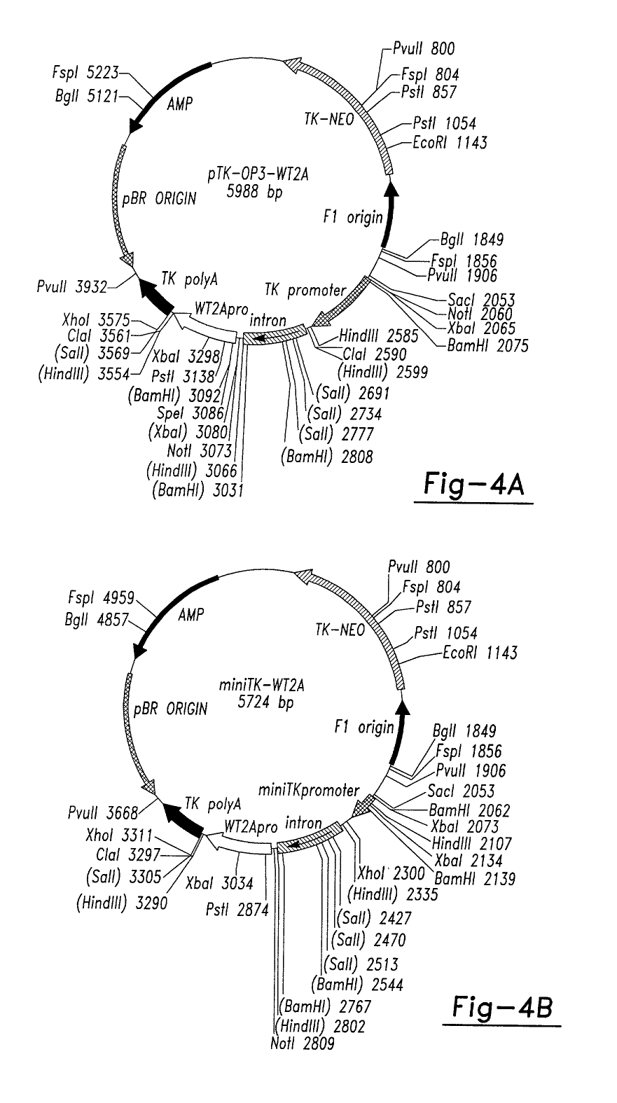

HEK-293 human (ATCC CRL-1573) cells were used as a model system for Polio virus 2A protease induced expression, since preliminary study indicated that 2A protease enhances expression of IRES containing genes in this cell line. HEK-293 cells were co-transfected with CMV-LacI—(constructed by applicant using techniques known to those skilled in the art) in combination with either one of the Polio virus 2A protease expression vectors PTK-OP3-WT2A, miniTK-WT2A, on PCIbb-LacI-Hyg (constructed by applicant on basis of vectors from Stratagene) as shown in FIGS. 4A-C, respectively. The LacI expression vector contained a hygromycin selectable marker, and the Polio virus 2A protease expression vector contained a neomycin selectable marker which enabled the isolation of clones resistant to both markers, presumably expressing both LacI repressor and Polio virus 2A proteins.

Analysis of Polio virus 2A protease expression

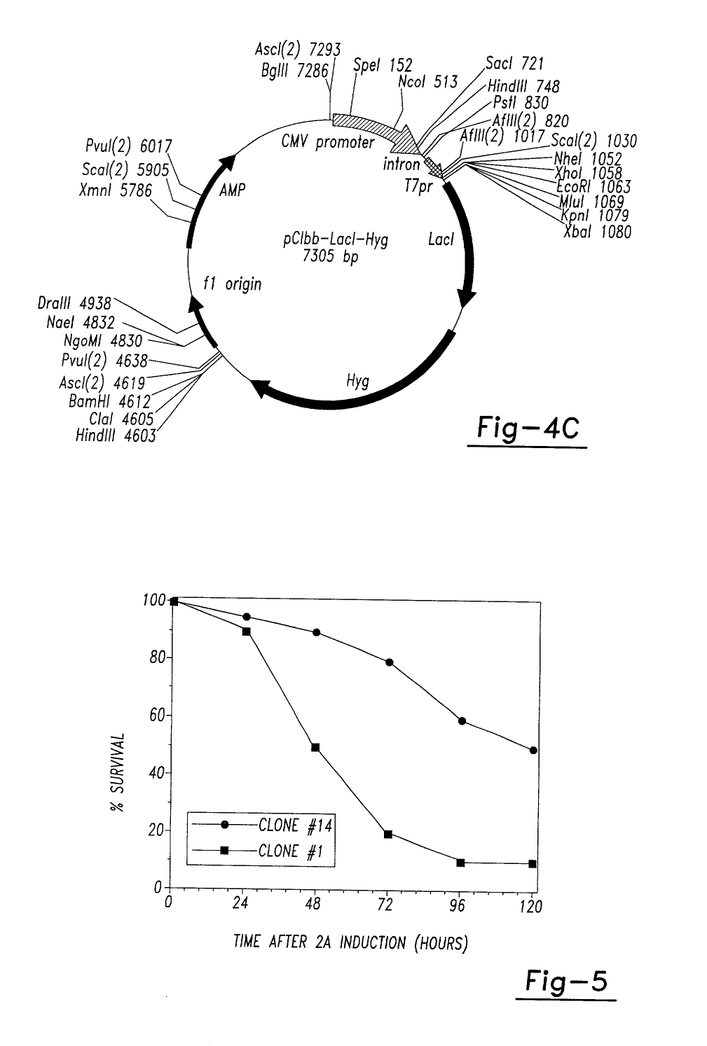

Death assay:—Resistant clones which grew after selection on hygromycin (50 μg/ml) and neomycin (500 μg/ml), were treated with IPTG (5mM for 48 h+5mM for further 48 h). Cells were then monitored for their viability and the clones that showed full mortality upon Polio virus 2A protease induction, presumably expressing the deleterious effect of the Polio virus 2A protease, were selected for for analysis. Two such clones were isolated, HEK-293 cells expressing Polio virus 2A protease under the control of a T, promotor (clone #14) and HEK-293 cells expressing the Polio virus 2A protease under the control of a miniTK promoter (clone #1 ) as shown in FIG. 5.

Analysis of 2A protease expression:—Direct analysis of the Polio virus 2A protease expression in HEK-293 miniTK#1 clones and HEK-293TK#14 clones after IPTG induction was not performed due to the lack of antibodies against the protein. Several currently available techniques can be used to measure changes in gene expression including Northern blot analysis, RNase protection assay, in situ hybridization, and reverse transcriptase polymerase chain reaction (RT-PCR). RT-PCR is a very sensitive method, and was used to monitor the induction of the mRNA encoding for Polio virus 2A protease in HEK-293 miniTK#1 clones following IPTG treatment. mRNA was prepared from HEK-293 parental cells and to HEK-293 miniTK-2A clones following treatment with IPTG at different time points. The RNAs were subjected to the RT-PCR reaction using Polio virus 2A protease specific oligonucleotides:

5′GCAACTACCATTTGGCCACTCAGGAA3′, (SEQ ID No: 28) and 5′GCAACCAACCCTTCTCCACCAGCAG3′and (SEQ ID No: 29).

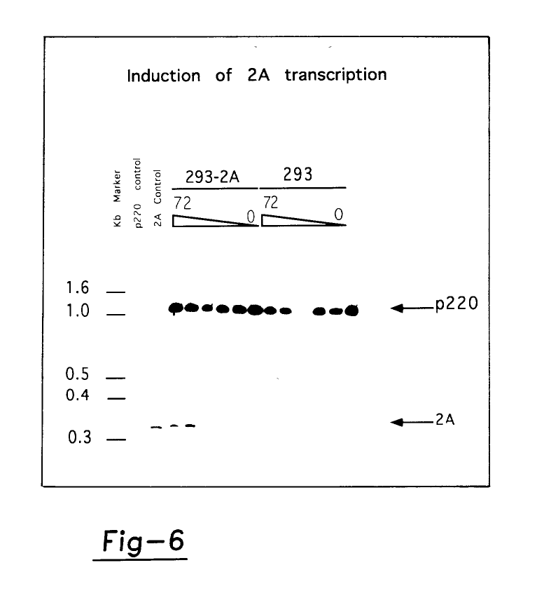

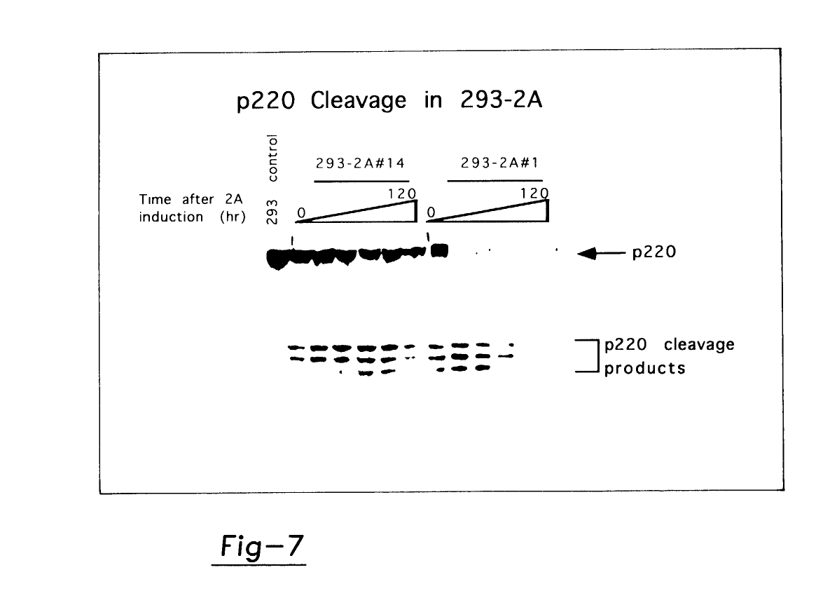

Polio virus 2A protease mRNA was not detected in HEK-293 parental cells, however it was induced following IPTG treatment and reached its highest level after 48 hours of IPTG treatment as shown in FIG. 6.

Analysis of 2A Protease activity

p220 cleavage:—A well characterized function of Polio virus 2A protease is the cleavage of the p220 protein (4Fγ translational factor), a component of the 40S ribosomal subunit. Cleavage of p220 yields three N-terminal cleavage products of 100-120KDa molecular weight due to post-translational modification. p220 and its cleavage products were identified by 7% SDS PAGE and Western blot analysis using polyclonal anti-p220 antibodies specifically directed against the N-terminal region p220 as shown in FIG. 6. FIG. 6 demonstrates such an analysis in which HEK-293 miniTK2A#1 clone and HEK, 293TK2A#14 clone were induced for Polio virus 2A protease expression to generate cleavage products of p220. As control, HEK-293 cell lysate was treated with Polio virus 2A protease produced by in vitro translation, and was found to generate identical cleavage products with the same mobility on 7% SDS PAGE as in the HEK-293 2A clones.

This system was used as the source of mRNA for polysomal fractionation. RDA analysis was performed using the protocol described above to identify genes whose translation was up-regulated by the effects of the Polio virus 2A protease. Table 4 summarizes the results of analyses performed according to the above-described method and genes isolated thereby.

Throughout this application various publications are referenced by citation and patents by number. Full citations for the publication are listed below. The disclosure of these publications in their entireties are hereby incorporated by reference into this application in order to more fully describe the state of the art to which this invention pertains.

The invention has been described in an illustrative manner, and it is to be understood the terminology used is intended to be in the nature of description rather than of limitation.

Obviously, many modifications and variations of the present invention are possible in light of the above teachings. Therefore, it is to be understood that within the scope of the appended claims, the invention may be practiced otherwise than as specifically described.

TABLE 1

| FRACTIONATION | |
|---|---|
| RNA associated with: | MEASURES AND IDENTIFIES |
| no fractionation | changes of transcript abundance |
| Total RNA | |
| Nuclear | Measures denovo synthesis of mRNA |
| Cytoplasmatic | Changes of transcript abundance |
| Cytoplasmatic/Nuclear | transport of mRNA from the nucleus |
| Nuclear/Cytoplasmatic | to the cytoplasm, increased or |
| | decreased stability of mRNA |
| Polyribosomal/subpoly | translationally controlled genes |
| ribosomal | |
| Rough Endoplasmic Reticulum | differences in the abundance of |
| Microsomes | transcripts encoding membrane and |
| membrane bound polysomes | secreted proteins |
| Cytoskeletal polyribosomes | differences in abundance of transcript |
| | encoding for nuclear proteins |
| mitochondrial | differences in the abundance of |
| | mRNA encoding mitochondrial |
| | proteins |
| Splicesome | differences in alternative splicing |

TABLE 2
Heat Shock Differential Gene Expression
Analysis with Polysomal Probes

| clone | Gene | Total | Polysomal |
|---|---|---|---|
| 13h04 | Pyruvate Kinase | No Change | Induced >>10 |
| 5b0g | Saposin | No Change | Induced >10 |
| 9f11 | Na,K-ATPase α-1 subunit | No Change | Induced ×4 |
| 1a04 | Thymopoietin α | No Change | Induced ×4 |
| 13h10 | Poly(ADP-ribose) polymerase | No Change | Induced ×5 |
| 7c09 | pM5 | Reduced ×2 | Induced >6 |
| 14e11 | Ubiquitin | Induced ×2 | Induced ×4 |
| 10c06 | Initiation Factor 4B | No Change | Induced ×4 |
| 1b09 | 90-kDa heat-shock protein | No Change | Induced >>10 |
| 1c06 | Acylamino acid-releasing | No Change | Induced >>10 |
| | enzyme | | |
| 1e09 | β-spectrin | Reduced ×2 | Induced ×5 |
| 3b04 | Elongation factor-1-gamma | No Change | Induced ×4 |
| 13a12 | Fibronectin | Induced ×2 | Induced ×10 |
| 7h12 | Cytochrome C reductase core [ | No Change | Induced >10 |
| 9d12 | Cytoskeletal γ-actin | No Change | Induced >6 |
| 13f09 | Protein disulfide isomerase | Reduced ×2 | Induced >10 |
| 9g12 | DAP5 | | Induced ×5 |

TABLE 3

| Statistics | Number of | |
|---|---|---|
| Probe | differentials | Fold induction |
| Total mRNA 4 hrs HS | 2 | 2 |
| Polysomal RNA 1 hr HS | 14 | 2-4 |
| | 8 | −8 |
| | 15 | >10 |
| | 37 | |
| Polysomal RNA 4 hrs HS | 13 | 2-4 |
| | 6 | −10 |
| | 18 | >10 |
| | 37 | |

TABLE 4
Translationally controlled genes
are identified by the 2A protease system

------------------------------------------------------------
A. Ribosomal proteins or proteins directly involved in
translation encoded by mRNAs containing 5′ TOP#

| | |
|---|---|
| S17 | gbM13932 |
| S9 | gb U14971 |
| EF-2 | gbM19997 |
| L27a | gb U14968 |
| L37a | gbL06499 |

(Meyuhas et al., 1996)
------------------------------------------------------------
B. Proteins encoded by mRNAs containing 5′TOP in their 5′
UTR

Laminin binding receptor
β1-tubulin gb J00314

------------------------------------------------------------
C. Gene with GC rich 5′UTR that regulates their
translation

spermidine synthase gbM34338
retinol binding protein 5′UTR X00129

------------------------------------------------------------
D. Unknown genes potenialy regulated by translation

| | | |
|---|---|---|
| EST gb1059051 | EST gb AA043162 | EST gbW76915 |
| EST gbT54424 | EST gb AA025896 | D 45282 |
| EST gbH15523 | EST gb R07358 | |
| EST gbW96821 | EST gb H83477 | |
| EST gbW99369 | EST T34436 | |

------------------------------------------------------------
E. Known genes that are potentially regulated by
translation (and may conatin IRES in their 5′ UTR)

mitochondrial hinge protein gbS61826
gp26L2 mitochondrial protein gp25L2
mRNA encoding a protein related to lysyl t-RNA
synthetase emb z31711
SAP14 human splicesosome gb U41371

------------------------------------------------------------

### REFERENCES CITED

Aiello et al., “Identification of multiple genes in bovine retinal pericytes altered by exposure to elevated levels of glucose by using mRNA differential display ” Proc. Natl. Acad. Sci. USA Vol.91, pp.6231-6235 (1994).

Bauer et al., “Identification of differentially expressed mRNA species by an improved display technique (DDRT-PCR)” Nucleic Acids Research, Vol. 21, No. 18 (1993).

Bharucha and Ven Murthy, “Characterization of Polysomes and Polysomal mRNAs by Sucrose Density Gradient Centifugation Followed by Immobilization in Polyacrylamide Gel Matrix” Methods in Enzymology Vol. 216, pp. 168-179 (1992).

Braun et al., “Identification of Target Genes for the Ewing's Sarcoma EWS/FLI Fusion Protein by Representational Difference Analysis” Molecular and Cellular Biology, Vol. 15, No. 8, pp. 4623-4630 (1995).

Cold Springs Harbor Laboratory Press, Cold Spring Harbor, N.Y., 1989

Davis et al., “Expression of a single transfected cDNA converts fibroblasts to myoblasts.” Cell 51:987-1000, 1987.

Diatchenko et al., “Suppression subtractive hybridization: A method for generating differentially regulated or tissue-specific cDNA probes and libraries” Proc. Natl Acad. Sci. Vol. 93, pp. 6025-6030 (1996).

Ehrenfeld, “Initiation of Translation by Picornavirus RNAs”, Translational Control Cold Spring Harbor Laboratory Press, pp. 549-573, 1996.

Hadman et al., “Modification to the differential display technique reduce background and increase sensitivity” Analytical Biochemistry 226:383-386 (1995).

Hanauske-Abel et al., “Detection of a sub-set of polysomal mRNAs associated with modulation of hypusine formation at the G1-S boundary. Proposal of a role for EIF-5A in onset of DNA replication.” FEBS Letters 386 pp. 92-98 (1995).

Hirama et al., “Direct Purification of Polyadenylated RNAs from Isolated Polysome Fractions” Analytical Biochemist, 155, pp. 385-390 (1986).

Hubank and Schatz, “Identifying differences in mRNA expression by representational difference analysis of cDNA” Nucleic Acids Research Vol. 22, No. 25, p. 5640-5648 (1994).

Jefferies et al., “Elongation Faction-1α mRNA Is Selectively Translated following Mitogenic Stimulation” The Journal of Biological Chemistry, Vol. 269, No. 6, pp. 4367-4372 (1994).

Liang and Pardee, “Differential Display of Eukaryotic Messenger RNA by Means of the Polymerase Chain Reaction” Science, Vol. 257, pp. 967-971 (1992).

Liang et al., “Distribution and cloning of Eukaryotic mRNAs by means of differential display: refinements and optimization” Nucleic Acids Research Vol. 21, No. 14, pp. 3269-3275 (1993).

Liang and Pardee, “Recent advances in differential display” Current Opinion in Immunology 7:274-280 (1995).

Linskens et al., Cataloging altered gene expression in young and senescent cells using enhanced differential display Nuc. Ac. Res. 23: 3244-3251 (1995).

Lisitsyn and Wigler, “Cloning the Differences Between Two Complex Genomes” Science Vol. 259, pp. 946-951 (1993).

Mach et al., “Isolation of a cDNA Clone Encoding S-Adenosylmethionine Decarboxylase” The Annual of Biological Chemistry, Vol. 261, No. 25, pp. 11697-11703 (1986).

Macejak et al., “Internal inition of translation mediated by the 5′ leader of a cellular mRNA” Nature, Vol. 353, pp. 990-94 (1991).

Mechler, “Isolation of messenger RNA from Membrane-Bound Polysomes” Methods in Enzymology, Vol. 152, pp. 241-253 (1987).

Menaker et al., “A Method for the Isolation of Rat Submandibular Salivary Gland Polysomes on Linear Sucrose Density Gradients” Analytical Biochemist 57, pp. 325-335 (1974).

Meyuhas et al., “Translational Control of Ribosomal Protein mRNAs in Eukaryotes” Translational Control, pp. 363-388 (1996).

Mountford et ale, “Internal ribosome entry sites and dicistronic RNAs in mammalian transgenesis” TIG Vol. 11. No. 5, pp. 179-184 (1995).

Ogishima et al., “Fractionation of Mammalian Tissue mRNAs by High-Performance Gel Filtration Chromatography” Analytical Biochemistry 138, pp. 309-313 (1984).

Oh et al., “Gene regulation: translational initiation by internal ribosome binding” Current Opinion in Genetics and development pp. 295-300(1993).

Pelletier et al., “Internal initiation of translation of eukaryotic mRNA directed by a sequence derived from poliovirus RNA” Nature, Vol. 334, pp.320-325 (1988).

Schena et al., “Quantitative Monitoring of Gene Expression Patterns with a Complementary DNA Microarray” Science. Vol. 270, pp. 467-470 1995).

Shen et al., “Identification of the Human Prostatic Carcinoma Oncogene PTI-1by Rapid Expression Cloning and Differential RNA Display” Proc. Natl. Acad. Sci. USA, Vol. 92, pp. 6778-6782 (1995).

Vagner et al. “Alternative Translation of Human Fibroblast Growth Factor 2 mRNA Occurs by Internal Entry of Ribosomes” Molecular and Cellular Biology, Vol. 15, No. 1, pp. 354 (1995).

Welsh et al., “Arbitrary primed PCR fingerprinting of RNA”, Nuc. Ac. Res. 20:4965-4970 (1992).

Zhao et al., “New primer strategy improves precision of differential display” Biotechniques 18: 842-850 (1995).

What is claimed is:

1. A method or process for identifying genes whose expression is responsive to a specific cue or cues including the steps of:
(a) applying a cue to an organism or tissue or cells;
(b) isolating specific cellular fractions from the tissues or cells subjected to the cue;
(c) extracting the mRNA from the cellular fractions; and
(d) differentially analyzing the mRNA samples in comparison with control samples not subjected to the cue to identify genes that have responded to the cue.

2. A method as set forth in claim 1, wherein the cue is a toxin or a chemical, or a pharmaceutical, or a mechanical stress, or an electric current, or a pathogen or a pathological condition, or a hormone, or a specific protein.

3. The method as set forth in claim 2, wherein said cue is further defined as chemically treating the cells, or irradiating the cells, or depriving the cells of oxygen.

4. A method as set forth in claim 2, wherein the cue is further defined as a stress-inducing element of unknown relationship to gene translation.

5. A method as set forth in claim l, wherein genes are identified at the translation level; genes regulated at the transcription level; genes regulated by RNA stability; genes regulated by mRNA transport rate between the nucleus and cytoplasm; genes regulated by differential splicing; and genes regulated by antisense RNA.

6. A method as set forth in claim 1, wherein the mRNA samples are farther fractionated into mRNA subfractions which are subjected to differential analysis to identify genes responsive to the cue at all levels of expression regulation as herein defined and to determine the abundance and direction of the response.

7. A method as set forth in claim 6, wherein the mRNA sample is fractionated into one or more subfractions from the group consisting essentially of cytoplasmic, nuclear, polyribosomal, sub polyribosomal, microsomal or rough endoplasmic reticulum, mitochondrial and splicesome associated mRNA.

8. A method as set forth in claim 1, wherein said differential analysis step is selected from the group consisting of differential display, representational differential analysis (RDA), suppressive subtraction hybridization (SSH), serial analysis of gene expression (SAGE), gene expression microarray (GEM), nucleic acid chip technology, oligonucleotide chip technology; DNA membrane arrays; direct sequencing and variations and combinations of these methods.

9. A method as set forth in claim 8, wherein said differential analysis step is further defined as identifying and measuring the genes regulated at the translation level.

10. A method as set forth in claim 8, wherein said differential analysis step is further defined as identifying and measuring the genes regulated at the transcription level.

11. A method as set forth in claim 8, wherein said differential analysis step is further defined as identifying and measuring the genes regulated by RNA stability.

12. A method as set forth in claim 8, wherein said differential analysis step is fisher defined as identifying and measuring the genes regulated by mRNA transport rate between the nucleus and the cytoplasm.

13. A method as set forth in claim 8 wherein said differential analysis step is further defined as identifying and measuring the genes regulated by differential splicing.

14. A method as set forth in claim 8, wherein said differential analysis step is further defined as identifying and measuring the genes encoding secreted and membrane proteins.

15. A method as set forth in claim 8, wherein said differential analysis step is further defined as identifying and measuring the genes encoding for nuclear proteins.

16. A method for identifying gene sequences coding for internal ribosome entry sites, said method comprising the steps of:
inhibiting 5′cap-dependant mRNA translation in a cell;
collecting a pool of mRNA from the cells; and
differentially analyzing the pool of mRNA to identify genes with sequences coding for internal ribosome entry sites.

17. A method as set forth in claim 16, wherein said inhibiting step is further defined as selecting for non-5′-cap dependent mRNA translation.

18. A method as set forth in claim 16, wherein said inhibiting step further includes the step of incorporating a gene coding for Polio virus 2A protease into the cell.

19. A method as set forth in claim 18; wherein said incorporation step is further defined as transforming the cell with a vector containing the gene coding for the Polio virus 2A protease.

20. A method as set forth in claim 18 including the step of controlling the expression of the gene coding for the Polio virus 2A protease.

21. A method as set forth in claim 16, wherein said analyzing step is further defined as differential display analysis.

22. A method as set forth in claim 16, wherein said analyzing step is further defined as representational difference analysis.

23. A method as set forth in claim 16, wherein said analyzing step is further defined as performing a gene expression microarray analysis.

24. A method as set forth in claim 16, including the further step of cloning genes identified as being translationally regulated.

25. A method as set forth in claim 16, wherein said analyzing step distinguishes between polysomal fractions that migrate in the same density individually or in a pool.

26. A method as set forth in claim 16, wherein said analyzing step distinguishes between nonpolysomal fractions individually or as a pool.

27. A method as set forth in claim 16, wherein said analyzing step distinguishes between stimulated polysomal and nonpolysomal fractions individually or in a pool.

28. A method as set forth in claim 16, wherein said analyzing step distinguishes between each of the polysomal and nonpolysomal fractions individually or in a pool compared to an unfractionated total RNA pool.
